(19) United States
(12) Patent Application Publication
(10) Pub. No.: US 2011/0313274 A1
(43) Pub. Date: Dec. 22, 2011

(54) Methods and apparatuses for 3D imaging in magnetoencephalography and magnetocardiography

(76) Inventor: Muralidhara Subbarao, Stony Brook, NY (US)

(21) Appl. No.: 12/924,959

(22) Filed: Oct. 9, 2010

Related U.S. Application Data
(60) Provisional application No. 61/397,958, filed on Jun. 19, 2010.

(51) Int. Cl. A61B 5/055

(52) U.S. Cl. 600/409

(57) ABSTRACT

This invention discloses methods and apparatuses for 3D imaging in Magnetoencephalography (MEG), Magnetocardiography (MCG), and electrical activity in any biological tissue such as neural/muscle tissue. This invention is based on Field Paradigm founded on the principle that the field intensity distribution in a 3D volume space uniquely determines the 3D density distribution of the field emission source and vice versa. Electrical neural/muscle activity in any biological tissue results in an electrical current pattern that produces a magnetic field. This magnetic field is measured in a 3D volume space that extends in all directions including substantially along the radial direction from the center of the object being imaged. Further, magnetic field intensity is measured at each point along three mutually perpendicular directions. This measured data captures all the available information and facilitates a computationally efficient closed-form solution to the 3D image reconstruction problem without the use of heuristic assumptions. This is unlike prior art where measurements are made only on a surface at a nearly constant radial distance from the center of the target object, and along a single direction. Therefore necessary, useful, and available data is ignored and not measured in prior art. Consequently, prior art does not provide a closed-form solution to the 3D image reconstruction problem and it uses heuristic assumptions. The methods and apparatuses of the present invention reconstruct a 3D image of the neural/muscle electrical current pattern in MEG, MCG, and related areas, by processing image data in either the original spatial domain or the Fourier domain.

### RELATED PROVISIONAL APPLICATION FOR PATENT

This patent application is a continuation of the following US Provisional Application for patent filed by this inventor:

M. Subbarao, “Methods and Apparatuses for Accurate and High-Resolution 3D Tomographic Imaging of Field Emission Sources in SPECT, PET, MRI, Magnetoencephalography, EEG, Inner Density Mapping of Earth, and Related Applications”, Provisional Application for Patent No. 61/397,958, filed Jun. 19, 2010.

This patent application provides full details of the description of the above invention related to Magnetoencephalography (MEG) and Magnetocardiography (MCG).

### FIELD OF INVENTION

This invention discloses novel methods and apparatuses for 3D imaging of electrical activity in human and animal organs such as the brain, the heart, and neural/muscle tissue in other parts of the body. Electrical activity in any neural/muscle tissue results in electrical current patterns that produce magnetic fields according to Biot-Savart's law. These magnetic fields can be measured using highly sensitive devices such as Superconducting Quantum Interference Devices (SQUIDS). Using such measured data, one can reconstruct a 3D image of the neural/muscle electrical current patterns. Imaging of such electrical activity of neural tissue in the brain is called Magnetoencephalography (MEG) and imaging of such electrical activity in the muscle tissue in the heart is called Magnetocardiography (MCG). Both MEG and MCG are extremely important in the medical diagnosis of neural/muscle illnesses.

### BACKGROUND

This invention discloses methods and apparatuses for 3D imaging in Magnetoencephalography (MEG), Magnetocardiography (MCG), and for 3D imaging of electrical activity in neural, muscle, or any biological tissue. This invention is based on Field Paradigm (FP) proposed recently by the author of this invention. Field Paradigm is a novel unified framework for several 3D medical imaging modalities including MEG and MCG. It is based on the Field Image Principle (FIP) stated as “the field intensity distribution in a 3D volume space uniquely determines the 3D density distribution of the field emission source and vice versa”. This principle has not been realized, recognized, or exploited in the past in 3D medical imaging. Theoretical soundness of this principle is easily verified. Experimental verification has been done on rudimentary cases through computer simulation.

Field Image Tomography (FIT) is a theoretical framework based on Field Paradigm that provides computational algorithms for reconstructing 3D density distribution of field emission sources from the measured 3D field image data. In MEG and MCG, electrical currents in neural/muscle elements act as sources of magnetic field in a 3D volume space.

Field Image Tomography, unlike techniques in prior art, is information-efficient in the sense that it exploits all available information that is useful for 3D image reconstruction. It is based on measuring field flux in a 3D volume space along up to 3 different directions instead of measurements on a 2D surface along a single direction at nearly fixed radial distance from the field emission source. In particular, field intensity produced by a small emission source decays with radial distance, and this characteristic of field propagation in a 3D volume space is exploited to determine the 3D density distribution of an emission source.

The data measured in the present invention captures all the available information and facilitates a computationally efficient closed-form solution to the 3D image reconstruction problem without the use of heuristic assumptions. This is unlike prior art where measurements are made only on a surface at a nearly constant radial distance from the center of the target object, and along a single direction. Therefore necessary, useful, and available data is ignored and not measured in prior art. Consequently, prior art does not provide a closed-form solution to the 3D image reconstruction problem and it uses heuristic assumptions.

In MEG the neural activity of the brain in different regions of the brain is characterized by the spatial electrical current density distribution. In MCG, the electrical activity in the heart muscle that makes the heart beat is characterized by the spatial electrical current density distribution. The current density distribution in both cases produces a magnetic field that is measured by high-sensitivity sensors such as SQUIDS (Superconducting Quantum Interference Devices). The measured values of the magnetic field intensity or its spatial derivatives are used to compute the 3D image of the current density distribution. This 3D image is useful in the diagnosis of many brain and heart ailments. Magnetic fields are preferable in medical imaging in comparison with electrostatic fields and X-rays because magnetic fields are harmless and pass through body tissue and bones without much attenuation and distortion. The present invention provides methods and apparatuses for MEG and MCG that are significantly more accurate and provide higher resolution 3D images thus improving the accuracy of diagnosis of ailments.

The methods and apparatuses in prior art in MEG and MCG rely solely on data measured on a surface and therefore they do not fully exploit all the available information. They ignore additional information available in the variation of magnetic field intensity with radial distance and direction in a 3D volume space around a target object. Therefore, prior art is unable to provide a closed-from solution to the 3D image reconstruction problem and must resort to heuristic assumptions. This results in lower accuracy and lower spatial and temporal resolution in the reconstructed image. It is an object of the present invention to exploit all the available information and provide efficient 3D imaging methods and apparatuses that have the advantages of higher accuracy and higher resolution.

This invention is based on measuring the magnetic field in an extended 3D volume space, not just on a surface at a nearly constant radial distance from the target object as in prior art. Magnetic field intensity measurements (or its derivatives) are made in a 3D volume space that extends substantially along the radial direction from the center of the target object. Further, magnetic field intensity measurement at each point is made along three mutually perpendicular directions. This has the effect of measuring a 3D vector field in a 3D space at different distances and along different directions.

Field Paradigm has been applied recently by the author of this invention to 3D medical imaging in two other cases: Single-Photon Emission Computed Tomography (SPECT) and Magnetic Resonance Imaging (MRI). A detailed description of the application of Field Paradigm to SPECT and MRI are provided in the following two U.S. patent applications filed by the author of this invention recently:
1. M. Subbarao, “Method and apparatus for high-sensitivity Single-Photon Emission Computed Tomography”, U.S. patent application Ser. No. 12/586,863, Date Sep. 29, 2009.
2. M. Subbarao, “Field Image Tomography for Magnetic Resonance Imaging”, U.S. patent application Ser. No. 12/658,001, Date Feb. 1, 2010.

The contents of the following U.S. patents on MEG and MCG in their entirety along with references therein are incorporated herein by reference:
1. S. J. Gott, A. D. Mascarenas, and R. C. Reineman, “High-Resolution Magnetoencephalography System, Components, and Method”, U.S. Pat. No. 7,197,352, B2, Date Mar. 27, 2007.
2. A. Ishiyama, Y. Ono, M. Murakami, “Magnetocardiograph”, U.S. Pat. No. 7,395,107, B2, Date: Jul. 1, 2008.

The prior art described in the above two patents and the references therein do not use a method that involves measuring magnetic field data in a 3D volume space that extends substantially along the radial direction, nor do they describe an apparatus that has a means for measuring the magnetic field around a biological tissue in a 3D volume space that extends substantially along the radial direction. Therefore, as explained earlier, the inventions in the above 2 patents suffer from many disadvantages such as being much less accurate leading to misdiagnosis of brain/heart related medical conditions.

### 3. OBJECTS AND ADVANTAGES

It is an object of the present invention to provide methods and apparatuses for 3D imaging in Magnetoencephalography, Magnetocardiography, and the 3D imaging of electrical activity in neural, muscle, or any biological tissue.

An advantage of the present invention is that it is information efficient, i.e. it measures and exploits all available information for 3D image reconstruction. Therefore it provides a more accurate 3D image in terms of spatial, temporal, and contrast or intensity resolution and therefore it improves the accuracy of medical diagnosis. Another advantage of the present invention is that, unlike prior art, it does not use heuristic assumptions and it provides a computationally efficient closed-form solution to the problem of 3D image reconstruction.

4. SUMMARY OF THE INVENTION

The present invention provides methods and apparatuses for 3D imaging of electrical activity in biological tissue in MEG and MCG. It includes a method of reconstructing a 3D image f(r1) that specifies a spatial density distribution of electrical currents in a biological tissue in a 3D volume space V1 at each point r1. This method comprises the following steps:
(a) Measuring up to three components of magnetic field intensity characteristics generated by the electrical currents in a 3D volume space V2 that in particular extends substantially along a radial direction pointing away from the approximate center of the biological tissue. The extension of the measurement volume space along the radial direction is unlike that in prior art and it facilitates capturing almost all available information for 3D image reconstruction. In particular, this captures information provided by the field decay along the radial direction which is not realized, recognized, or exploited in prior art. This measured data is recorded as a function of measurement position r2 in a 3D volume space V2 as g(r2).
(b) Determining a system matrix H(r1,r2) that specifies magnetic field intensity characteristics at point r2 produced by an electric current source of unit length located at point r1. This field matrix H(r1,r2) is determined based on magnetic field generation characteristics as well as magnetic field measurement apparatus characteristics. It also incorporates conditions to satisfy Kirchoff's current law at each point (that total electric current flowing into a small volume element of tissue is zero).
(c) Setting up a vector-matrix equation g(r2)=H(r1,r2) f(r1)+n(r2) where n(r2) represents noise in measured data at point r2; and
(d) Solving the vector-matrix equation g(r2)=H(r1,r2) f(r1)+n(r2) and estimating a solution f1(r1) for the 3D image f(r1) using a method that reduces the effect of noise n(r2) so that estimated solution f1(r1) is close to desired solution f(r1).

The biological tissue in the above method can be that of a mammal brain including humans and some animals. In this case the method is for magnetoencephalography or MEG. If the biological tissue is that of a mammal/human heart then the method is for magnetocardiography or MCG. In this method, the measured magnetic field characteristics can include the spatial derivatives of the magnetic filed. Measuring spatial derivatives of magnetic field intensity instead of actual intensity eliminates the effects of the background magnetic field of the earth.

In the above method, the vector-matrix equation g(r2)=H(r1,r2) f(r1)+n(r2) may involve the convolution of H(r1,r2) and f(r1). In that case, Step (d) above is carried-out in the Fourier domain by computing the Fourier transforms of g(r2), and H(r1,r2), and f1(r1) is computed using inverse Fourier transform and inverse filtering in the Fourier domain.

The present invention includes an apparatus for measuring magnetic field intensity characteristics around a biological tissue enclosed in a 3D volume space V1. This apparatus could be used in applications such as magnetoencephalography and magnetocardiography. This apparatus comprises a means for measuring and recording magnetic field intensity characteristics of magnetic field generated by an electric current pattern in a biological tissue. The electric current pattern in the biological tissue can have a 3D spatial density distribution of f(r1). This means for measuring magnetic field intensity characteristics must specifically be capable of measuring at a set of points r2 in a 3D volume space V2. This volume space V2 in particular extends substantially along the radial direction pointing away from the approximate center of the biological tissue. The measured data is recorded as a function of position r2 of points in the 3D volume space V2 as g(r2).

The apparatus described above may further include the following components:
(a) A means such as a digital computer for setting up a vector-matrix equation of type g(r2)=H(r1,r2) f(r1)+n(r2) where H(r1,r2) represents a system matrix. This matrix H(r1,r2) specifies magnetic field intensity at point r2 produced by an electric current density of unit strength located at point r1. H(r1,r2) also includes conditions for satisfying the Kirchoff's current law that the total current flowing into any volume element is zero. Further, the term n(r2) represents noise in measured data at point r2.
(b) A means for solving the vector-matrix equation g(r2)=H(r1,r2) f(r1)+n(r2) and estimating a solution f1 (r1) for the 3D image f(r1) using a method that reduces the effect of noise n(r2) so that estimated solution f1 (r1) is close to desired solution WO.

In the apparatus described above, the means for solving the vector-matrix equation g(r2)=H(r1,r2) f(r1)+n(r2) can further include a means for computing Discrete Fourier Transforms (DFT) and inverse filtering. This part can be used in solving the vector-matrix equation g(r2)=H(r1,r2) f(r1)+n(r2) in the Fourier domain.

The apparatus above can be made in a shape suitable for the case when the biological tissue is a brain being imaged in Magentoencephalography. It can also be made in a shape suitable for the case when the biological tissue is a heart being imaged in Magentocardiography. The apparatus can further include a digital computer and a display monitor for displaying images.

### 5. BRIEF DESCRIPTION OF DRAWING

FIG. 1 shows a certain embodiment of the apparatus of the present invention. A 3D array of magnetic field measurement sensors 101 is provided. This sensor array contains a large number of small sensor elements such as SQUIDS distributed in a 3D volume space. At each point, the sensor elements in the 3D array can measure the magnetic field intensity or its spatial derivatives along three mutually perpendicular directions. This sensor array has a shape such that it surrounds the biological object such as the human brain or heart that needs to be imaged. In particular, unlike prior art, the sensor elements are distributed along all directions including substantially along the radial direction 105 that points away from the center of the object of study. In prior art, the sensor elements are typically restricted to lie on a 2D surface that covers the object of study but the surface itself is thin along the radial direction 105 and so the sensor elements are not distributed along the radial direction. This is a critical and distinguishing feature of the present invention because it is the distribution of the sensors along the radial direction 105 that captures the additional information necessary for 3D image reconstruction of electrical activity in the biological object. The biological object 102 is shown to be surrounded by the 3D sensor array. This object will be the human brain in MEG, and human heart in MCG. It can be any biological object such as an animal tissue with electrical activity. The 3D sensor array is connected to computer 103 for processing measured data. The computer 103 is also connected to a display device 104 such as a computer monitor for displaying the reconstructed 3D images for viewing by humans.

FIG. 2 shows the geometry of the field emission source or the biological tissue 403 at position r, magnetic field measurement sensor 402, one element of magnetic field sensor at position r′, field emission source object or biological object 401, and volume 404 in which the field emission source is distributed.

FIG. 3 shows a certain embodiment of the apparatus of the present invention for magnetocardiography (MCG). This Figure is similar to FIG. 1 but the biological object in this case is the human chest for imaging the heart. A 3D array of magnetic field measurement sensors 301 is provided. This sensor array contains a large number of small sensor elements such as SQUIDS distributed in a 3D volume space that extends substantially along the radial direction 305. The biological object 302 is shown to be surrounded by the 3D sensor array. The 3D sensor array is connected to computer 303 for processing measured data. The computer 303 is also connected to a display device 304 such as a computer monitor for displaying the reconstructed 3D images for viewing by humans.

FIG. 4 shows a flow-chart of the method of the present invention.

### 6. DESCRIPTION OF THE INVENTION

This invention discloses novel methods and apparatuses for 3D imaging in MEG and MCG. A detailed description of the methods and apparatuses are presented in this section.

The present invention is based on a new theory not found in prior art. It is based on the Field Paradigm. Therefore, the theoretical basis of the present invention is presented with concrete mathematical derivations for 3D imaging in MEG and MCG.

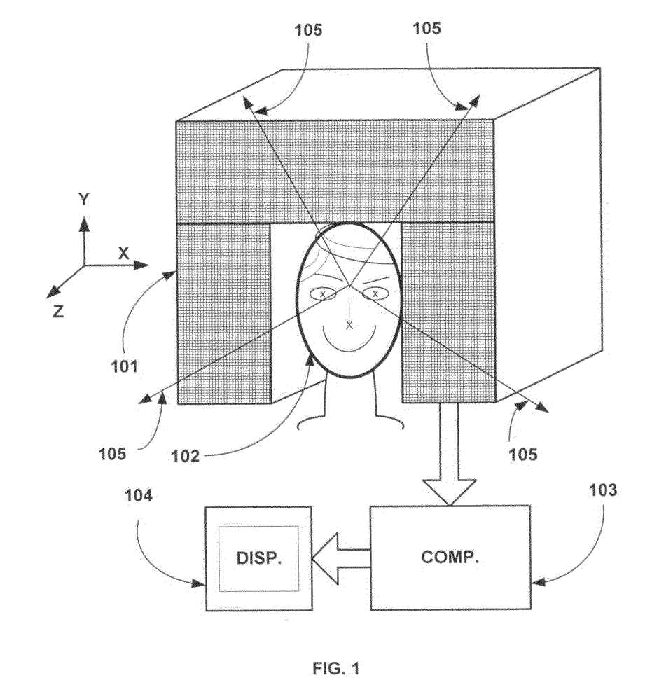

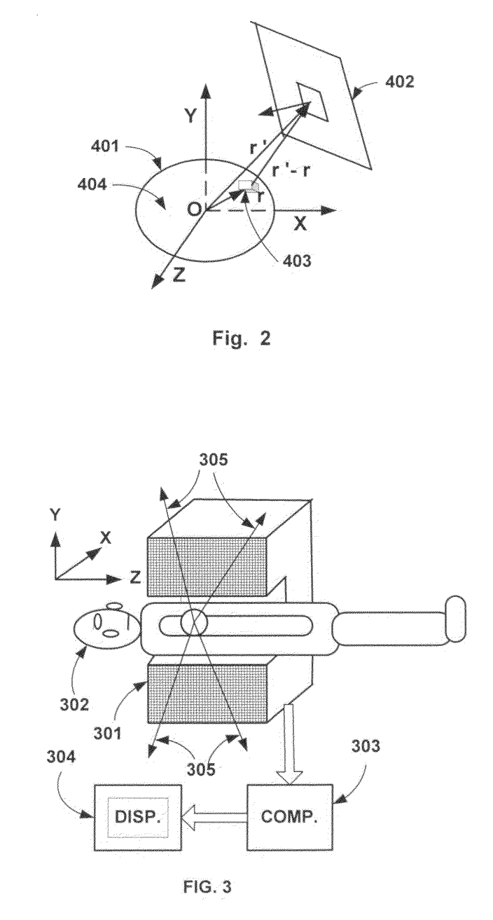

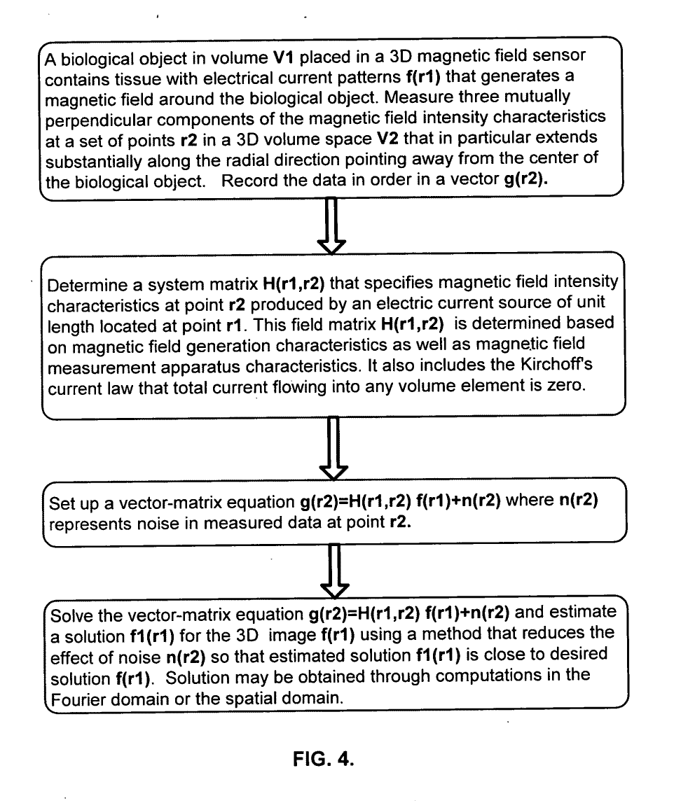

Consider an object to be imaged with biological tissue in which an electrical current pattern is present. For example, in MEG, this object would be the neural tissue in a brain and in MCG this would be the muscle tissue in a heart. The case of a human head placed in an MEG apparatus is shown in FIG. 1. This object 102 occupies a certain 3D volume space V1 inside a cubical volume, say with an approximate dimension of 200 mm on each side. In FIG. 2, the object is shown as 401 and volume V1 is shown as 404. This cubical volume can be thought of as being made up of small cubic volume elements or voxels of dimensions 1 mm on each side. In general, the length, width, and height of such voxels can be specified as dx, dy, dz along x, y, and z axes, at each point. The position of any voxel occupied by the object with coordinates (x, y, z) is specified by a 3D position vector r and the unit vectors i,j,k, along x,y,z, axes respectively as

r=ix+jy+kz. (1)

For example, see FIG. 2 where the voxel 403 and its position r are shown.

Let the current density in each voxel be

J(r)=iJₓ(r)+jJ_{y}(r)+kJ_{z}(r). (2)

Let the measured magnetic field B(r′) at a point r′ be represented by a 3D vector with unit vectors i, j, k, along the x, y, and z axes respectively by

B(r′)=Bₓ(r′)+jB_{y}(r′)+kB₃(r′) (3)

and

r′=ix′+jy′+kz′. (4)

See FIG. 2 which shows r′ and the sensor 402 for measuring the magnetic field. The problem in MEG/MCG is, given B(r′) measured at a large number of points r′ spread out along all dimensions in a 3D volume space, and measured along 3 mutually perpendicular directions, estimate the 3D current density function J(r). According to Biot-Savart law, the magnetific field dB₁(r′) at r′ due to the x-axis component of current (note: dA=dy dz is the area of one face of the voxel):

Iₓ(r)=Jₓ(r)dA=Jₓ(r)dy dz (5)

in a single voxel of size or volume

dV=dx dy dz (6)

at r is given by

$$d {B}_{1} ( {r}^{′} ) = ( i {J}_{x} ( r ) dy dz dx ) \times ( c \frac{( {r}^{′} - r )}{{\vert {r}^{′} - r \vert}^{3}} ) \qquad ( 7 )$$

where constant c is related to the magnetic permeability constant μ₀ as

$$c = ( \frac{{μ}_{0}}{4 π} ) . \qquad ( 8 )$$

Therefore, the total magnetic field due to only the x-component of current is given by the volume integral

$${B}_{1} ( {r}^{′} ) = c {\int }_{V} ( i {J}_{x} ( r ) ) \times \frac{( {r}^{′} - r )}{{\vert {r}^{′} - r \vert}^{3}} d V \qquad ( 9 )$$

### DEFINE

$$H ( {r}^{′} - r ) = c \frac{( {r}^{′} - r )}{{\vert {r}^{′} - r \vert}^{3}} . \qquad ( 10 )$$

Then we obtain

$${B}_{1} ( {r}^{′} ) = {\int }_{V} ( i {J}_{x} ( r ) ) \times H ( {r}^{′} - r ) d V . \qquad ( 11 )$$

Eq. (11) above is a convolution expression. It can be solved easily in the Fourier domain if the y and z components of the current are zero everywhere.

Similarly, the total magnetic fields due to the y and z components of the current are

$$\begin{matrix}{B}_{2} ( {r}^{′} ) = {\int }_{V} ( j {J}_{y} ( r ) ) \times H ( {r}^{′} - r ) d V \\ and & ( 12 ) \\ {B}_{3} ( {r}^{′} ) = {\int }_{V} ( k {J}_{z} ( r ) ) \times H ( {r}^{′} - r ) d V & ( 13 )\end{matrix}$$

respectively. Therefore the total magnetic field due to all the three current components is

B(r′)=B₁(r′)+B₂(r′)+B₃(r′). (14)

Let

B(r′)=iBₓ(r′)+jB_{y}(r′)+kB₃(r′). (15)

It can be expressed as

$$B ( {r}^{′} ) = {\int }_{V} J ( r ) \times H ( {r}^{′} - r ) d V \qquad ( 16 )$$

Or, in component form, the three components of B(r′) are

$$\begin{matrix}{B}_{x} ( {r}^{′} ) = {\int }_{V} ( {J}_{y} ( r ) {H}_{z} ( {r}^{′} - r ) - {J}_{z} ( r ) {H}_{y} ( {r}^{′} - r ) ) d V & ( 17 ) \\ {B}_{y} ( {r}^{′} ) = {\int }_{V} ( {J}_{z} ( r ) {H}_{x} ( {r}^{′} - r ) - {J}_{x} ( r ) {H}_{z} ( {r}^{′} - r ) ) d V & ( 18 ) \\ {B}_{z} ( {r}^{′} ) = {\int }_{V} ( {J}_{x} ( r ) {H}_{y} ( {r}^{′} - r ) - {J}_{y} ( r ) {H}_{x} ( {r}^{′} - r ) ) d V . & ( 19 )\end{matrix}$$

Measuring the three mutually perpendicular components Bₓ(r′), B_{y}(r′), B₃(r′) of B(r′) is a key step in the method of the present invention. These measurements are made at a large number of points r′ (or r2) distributed in a 3D volume space V2. These measured values are related to the unknown J(r) and the point spread function H(r′−r) by Equation (16) or Eqns. (17), (18), and (19). These equations have the form of a convolution expression which can be solved along with additional constraints for J(r) in the Fourier domain. For example, let the respective Fourier transforms be represented by lower case letters. Then we obtain,

u=(uₓ,u_{y},u_{z}) (20)

which denotes the spatial Fourier frequencies. Further Eqns. (17) to (19) have the following equivalent equations in the Fourier domain:

bₓ(u)=j_{y}(u)h_{z}(u)−j_{z}(u)h_{y}(u) (21)

b_{y}(u)=j_{z}(u)hₓ(u)−jₓ(u)h_{z}(u) (22)

b_{z}(u)=jₓ(u)h_{yl (u)-j}_{y}(u)hₓ(u) (23)

In matrix form, we may write the above equations as

$$\begin{matrix}b ( u ) ≡ {H}^{′} ( u ) j ( u ) \\ where & ( 24 ) \\ b ( u ) ≡ [ \begin{matrix}{b}_{x} ( u ) \\ {b}_{y } ( u ) \\ {b}_{z} ( u )\end{matrix} ] & ( 25 ) \\ {H}^{′} ( u ) = [ \begin{matrix}0 & {h}_{z} ( u ) & - {h}_{y} ( u ) \\ - {h}_{z} ( u ) & 0 & {h}_{x} ( u ) \\ {h}_{y} ( u ) & - {h}_{x} ( u ) & 0\end{matrix} ] & ( 26 ) \\ j ( u ) ≡ [ \begin{matrix}{j}_{x} ( u ) \\ {j}_{y} ( u ) \\ {j}_{z} ( u )\end{matrix} ] & ( 27 )\end{matrix}$$

The equation

b(u)=H′(u)j(u) (28)

is under-constrained and cannot be solved uniquely unless an additional constraint is employed. The determinant of H′(u) can be verified to be zero.

The additional constraint we can use is the Krichoff's current law at each voxel. The electric charge in a voxel is conserved and therefore the total charge or current flowing into any voxel from all neighboring voxels must be zero. In the discrete domain this condition can be expressed for a voxel with dimensions δx×δy×δz and δ=δx=δy=δz by

Jₓ(r+iδ)−Jₓ(r−iδ)+J_{y}(r+jδ)−J_{y}(r−jδ)+J_{z}(r+kδ)−J_{z}(r−kδ)=0 (29)

In the continuous domain, this condition can be expressed by

divergenceJ(r)=0, or (30)

$$∇ \cdot J ( r ) = \frac{\partial {J}_{x} ( r )}{\partial x} + \frac{\partial {J}_{y} ( r )}{\partial y} + \frac{\partial {J}_{z} ( r )}{\partial z} = 0. \qquad ( 31 )$$

In the Fourier domain, using the Fourier transforms of derivatives, the constraint divergence J(r)=0 becomes

(−2πi)(iₓjₓ(u)+u_{y}j_{y}(u)+u_{z}j_{z}))=0 (32)

where i is the imaginary square root of −1. Using the above equation and the equation b(u)=H′(u)j(u), we can solve for jₓ(u), j_{y}(u), j_{z}(u) and therefore J(r). We can add the above equation to each of the row in the matrix equation b(u)=H′(u)j(u) to obtain an equivalent solvable equation to be

$$\begin{matrix}b ( u ) = H ( u ) j ( u ) \\ where & ( 33 ) \\ H ( u ) = [ \begin{matrix}{u}_{x} & {u}_{y} + {h}_{z} ( u ) & {u}_{z} - {h}_{y} ( u ) \\ {u}_{x} - {h}_{z} ( u ) & {u}_{y} & {u}_{z} + {h}_{x} ( u ) \\ {u}_{x} + {h}_{y} ( u ) & {u}_{y} - {h}_{x} ( u ) & {u}_{z}\end{matrix} ] & ( 34 )\end{matrix}$$

The solution of course is

j(u)≡H⁻¹(u)b(u) (35)

and

J(r)=F⁻¹{j(u)} (36).

where F⁻¹{Ku)} is the inverse Fourier transform of j(u). Thus Eq. (36) provides a closed-form solution in the Fourier domain. Having measured the three mutually perpendicular components Bₓ(r′), B_{y}(r′), B₃(r′) of B(r′), in the Fourier domain, the solution for the 3D image reconstruction problem is computed using Eqns. (35) and (36). These equations (33) to (36) in fact constitute a theoretical proof of the existence of the unique solution and a proof of the Field Image Principle, in the case of MEG and MCG. Once the existence of a unique solution is established as done here, either Fourier or spatial domain techniques may be used to solve the problem.

It is also possible to compute a solution in the spatial domain by writing equations in the discrete spatial domain that are equivalent to Eq. (16) or Eqns. (17), (18), (19), and Eq. (29). In this case one obtains a large system of linear equations expressed as a vector matrix equation of the form

g(r′)=H(r′,r)f(r)+n(r′) (37.1)

or, setting r1=r and r2=r′, Eq. (37.1) becomes

g(r2)=H(r1, r2)f(r1)+n(r2) (37.2).

In Eqs. (37.1) and (37.2), g(r′) and g(r2) denote a column vector of measured magnetic field intensity data or derivatives of the intensity corresponding to B(r′); H(r′,r) and H(r1,r2) denote a system matrix related to the point spread function (e.g. see in Eq. (10)) and Kirchoff's current law (see Eq. (29)); f(r) and f(r1) denote a column vector of unknown current density distribution corresponding to J(r) in Eq. (16) that needs to be solved for; and, n(r′) and n(r2) denote noise in the measured data. The system matrix H(r1,r2) is affected by sensor characteristics and geometry. Eq. (37.2) can be solved using any one of many available techniques in the literature. This solution is carried out in a computer 103 shown in FIG. 1, and then the reconstructed 3D image f(r1) or J(r) is displayed on a computer monitor or display 104.

A regularized inverse filter such as a Weiner filter or a spectral filter can also be used for computing f using the above equations.

Although Equation (36) provides a solution, in practice, due to the interference of the Earth's magnetic field with the magnetic field produced by electric current in a body tissue, a minor change is needed. The effect of Earth's magnetic field can be cancelled by measuring spatial derivatives of B(r′) in Equations (16) to (19) instead of just B(r′) and specified by its components. SQUIDS (Superconducting Quantum Interference Devices) can directly measure such spatial derivatives of magnetic fields. For example, the following equations can be used instead of Eqs. (17) to (19):

$$\begin{matrix}\frac{\partial }{\partial x} {B}_{x} ( {r}^{′} ) = \frac{\partial }{\partial x} {\int }_{V} ( {J}_{y} ( r ) {H}_{z} ( {r}^{′} - r ) - {J}_{z} ( r ) {H}_{y} ( {r}^{′} - r ) ) d V & ( 38 ) \\ \frac{\partial }{\partial y} {B}_{y} ( {r}^{′} ) = \frac{\partial }{\partial y} {\int }_{V} ( {J}_{z} ( r ) {H}_{x} ( {r}^{′} - r ) - {J}_{x} ( r ) {H}_{z} ( {r}^{′} - r ) ) d V & ( 39 ) \\ \frac{\partial }{\partial z} {B}_{z} ( {r}^{′} ) = \frac{\partial }{\partial z} {\int }_{V} ( {J}_{x} ( r ) {H}_{y} ( {r}^{′} - r ) - {J}_{y} ( r ) {H}_{x} ( {r}^{′} - r ) ) d V & ( 40 )\end{matrix}$$

Therefore, taking the Fourier transform on either side of Eqs. (38) to (40), using the property of the Fourier transforms of derivatives, and using suitable notation, the following equations can be derived:

bₓ^{x}(u)=(−i2πuₓ)(j_{y}(u)h_{z}(u)−j_{z}(u)h_{y}(u)) (41)

b_{y}^{y}(u)=(−i2πu_{y})(j_{z}(u)hₓ(u)−jₓ(u)h_{z}(u)) (42)

b_{z}^{z}(u)=(−i2πu_{z})(jₓ(u)h_{y}(u)−j_{y}(u)hₓ(u)) (43)

The above equations along with Eq. (32) can be used to solve for J(r)=F⁻¹ {j(u)}. Equations equivalent to (41) to (43) and (32) can be written in the spatial domain in the form g(r2)=H(r1,r2) f(r1)+n(r2) and solved.

An important advantage of the present invention is the measurement and exploitation of all available information. This results in better accuracy and higher spatial and temporal resolution of reconstructed 3D tomographic images. A novel feature of this invention is the measurement of magnetic field pattern not just on a thin surface but in a 3D volume space that extends substantially along the radial direction in addition to two other mutually perpendicular directions. At each point in the 3D volume space, measurements should be made along 3 mutually perpendicular directions. The method of the present invention processes this 3D volume data to reconstruct 3D current patterns that reveal the location and intensity of electrical activity in the brain or heart or any target object.

### 6.1 Apparatuses of the Invention

The present invention includes an apparatus for measuring magnetic field intensity characteristics around a biological tissue enclosed in a 3D volume space V1. This apparatus could be used in applications such as magnetoencephalography (MEG) and magnetocardiography (MCG). A certain embodiment of this apparatus is shown in FIG. 1.

The apparatus includes a means 101 for measuring magnetic field intensity characteristics of magnetic fields. For example, it could be a 3D array of SQUIDS surrounding the biological object 102. It could also be a 2D array of SQUIDS that is moved to cover a 3D volume space around the biological object and measure the magnetic field characteristics at different points in the 3D volume space. The sensor array 101 measures the magnetic field generated by the electric current pattern in the biological tissue 102 such as the animal/human brain/heart. The electrical current pattern in the biological tissue can have a 3D spatial density distribution of f(r1) corresponding to j(r). The sensor array for measuring magnetic field intensity characteristics must specifically be capable of measuring at a set of points r2 in a 3D volume space V2 around the biological object 102. This volume space V2 in particular extends substantially along the radial direction pointing away from the approximate center of the biological tissue/object. This is the key distinguishing feature of the apparatus of the present invention that is not found in prior art. This feature was not introduced in prior art as the field paradigm and the field image principle disclosed here were not known in prior art. This apparatus is further capable of recording measured data as a function of position r2 of points in the 3D volume space V2 as g(r2) corresponding to B(r).

The apparatus described above may further include a means such as a digital computer 103 for setting up a vector-matrix equation of type g(r2)=H(r1,r2) f(r1)+n(r2) where H(r1,r2) represents a system matrix. This matrix H(r1,r2) specifies magnetic field intensity at point r2 produced by an electric current density of unit strength located at point r1. Further, the term n(r2) represents noise in measured data at point r2. This computer is further provided with a software or hardware means for solving the vector-matrix equation g(r2)=H(r1,r2) f(r1)+n(r2) and estimating a solution f1(r1) for the 3D image f(r1). This means for estimating the solution for f(r1) is such that it reduces the effect of noise n(r2) so that estimated solution f1(r1) is close to desired solution f(r1). Many standard systems are available for this purpose including those based on regularization.

In the apparatus described above, the means for solving the vector-matrix equation g(r2)=H(r1,r2) f(r1)+n(r2) can further include a means for computing Discrete Fourier transforms and inverse filtering. This part can be used in solving the vector-matrix equation g(r2)=H(r1,r2) f(r1)+n(r2) in the Fourier domain.

The apparatus above can be made in a shape suitable for different biological objects. For example, in the case of human brain and MEG, the sensor array 101 can have a shape suitable for enclosing a human head. It may take the shape of a very thick helmet. The apparatus can also have a shape suitable for surrounding a human chest for MCG. In this case, electrical activity of the heart can be measured. The 3D sensor array in this case may have the shape of a thick cylinder.

### 6.2 METHODS OF THE INVENTION

The present invention provides methods for 3D imaging of electrical activity in any biological tissue such as brain, heart, etc, in MEG, MCG, and related applications. It includes a method of reconstructing a 3D image f(r1) that specifies a spatial density distribution J(r) of electrical currents in a biological tissue in a 3D volume space V1 at each point r1. This method comprises the following steps.

In the first step, three components of magnetic field intensity characteristics in Equations (17) to (19) or Eqns. (38) to (40) are measured. These magnetic field intensity characteristics are produced in a 3D volume space V2 by the electrical current patterns in the biological tissue. The measurements are made in the 3D volume space V2 at a set of points r2 that in particular extends substantially along a radial direction pointing away from the approximate center of the biological tissue. The extension of the measurement volume space along the radial direction is unlike that in prior art and it facilitates capturing almost all available information for 3D image reconstruction. In particular, this captures information provided by the field decay along the radial direction which is not realized, recognized, or exploited in prior art. This measured data is recorded as a function g(r2) of measurement position r2 in a 3D volume space V2.

In the next step, a system matrix H(r1,r2) is determined which specifies magnetic field intensity characteristics at point r2 produced by an electric current source of unit length located at point r1. H(r1,r2) also includes Kirchoff's current law in Eq. (29). This field matrix H(r1,r2) is determined based on magnetic field generation characteristics as well as magnetic field measurement apparatus characteristics. For example, Equation (10) and Eq. (29) together specify H(r1,r2) in a simple case. Next the unknown current density J(r) in Equation (16) is represented by a vector f(r1) by lexicographic ordering of J(r) based on row, column, and frame order of data. Then the vector-matrix equation g(r2)=H(r1,r2) f(r1)+n(r2) is set up where n(r2) represents noise in measured data at point r2. This equation effectively represents the discrete version of Equation (16) or Eqns. (17) to (19) and Eqn. (29). Alternately, in order to cancel the effect of earth's magnetic field, this equation g(r2)=H(r1,r2) f(r1)+n(r2) can effectively represent the discrete equivalent of Equations (38) to (40) and Eq. (29).

The last step in the method of the present invention is to solve the vector-matrix equation g(r2)=H(r1,r2) f(r1)+n(r2) and estimate a solution f1(r1) for the 3D image f(r1). This solving is carried-out using a method that reduces the effect of noise n(r2) so that the estimated solution f1 (r1) is close to desired solution f(r1). Any standard technique that reduces the effect of noise such as a regularization technique can be used.

The biological tissue in the above method can be that of a mammal brain including humans and some animals. In this case the method is for magnetoencephalography or MEG. If the biological tissue is that of a mammal/human heart then the method is for magnetocardiography or MCG. In this method, the measured magnetic field characteristics can include the spatial derivatives of the magnetic filed as in Equations (38) to (40). Measuring spatial derivatives of magnetic field intensity instead of actual intensity eliminates the effects of the background magnetic field of the earth.

In the above method, the vector-matrix equation g(r2)=H(r1,r2) f(r1)+n(r2) may involve the convolution of H(r1,r2) and f(r1). For example, Equations (16) or (17) to (19) are convolutions. In that case, solving the equation g(r2)=H(r1,r2) f(r1)+n(r2) can be done in the Fourier domain. The equivalent Fourier domain equations are given by Eqns. (32) to (36). When the spatial derivatives on the magnetic field are used to cancel the effect of the earth's magnetic field, then Equations (41) to (43), and Eqn. (32) can be used. In this method, forward and inverse discrete Fourier transforms are computed using the Fast Fourier Transform algorithm. A regularization approach such as the Weiner filtering approach can be used in the Fourier domain method of solving the equation g(r2)=H(r1,r2) f(r1)+n(r2). This reduces the effect of noise in the estimated solution.

### 8. CONCLUSION, RAMIFICATIONS AND SCOPE OF THE INVENTION

The present invention provides a novel approach based on Field Paradigm for MEG, MCG, and related applications. The present invention is also applicable to objects that are not biological objects. While the description here of the methods, apparatuses, and applications contain many specificities, these should not be construed as limitations on the scope of the invention, but rather as exemplifications of preferred embodiments thereof. Further modifications and extensions of the present invention herein disclosed will occur to persons skilled in the art to which the present invention pertains, and all such modifications are deemed to be within the scope and spirit of the present invention as defined by the appended claims and their legal equivalents thereof.

What is claimed is:

1. A method of reconstructing a 3D image f(r1) that specifies a spatial density distribution of electrical currents in a biological tissue in a 3D volume space V1 at each point r1, said method comprising the steps of:
(a) measuring up to three components of magnetic field intensity characteristics generated by electrical currents in said biological tissue, measurement being made in a 3D volume space V2 that in particular extends substantially along a radial direction pointing away from the approximate center of said biological tissue thereby capturing almost all available information for 3D image reconstruction, and recording this measured data as a function of position r2 of points in said 3D volume space V2 as g(r2);
(b) determining a system matrix H(r1,r2) that specifies magnetic field intensity characteristics at point r2 produced by an electric current source of unit strength located at point r1 and Kirchoff's current law, said field matrix H(r1,r2) determined based on magnetic field generation characteristics, Kirchoff's current law, as well as magnetic field measurement apparatus characteristics;
(c) setting up a vector-matrix equation g(r2)=H(r1,r2) f(r1)+n(r2) where n(r2) represents noise in measured data at point r2; and
(d) solving said vector-matrix equation g(r2)=H(r1,r2) f(r1)+n(r2) and estimating a solution f1(r1) for said 3D image f(r1) using a method that reduces the effect of noise n(r2) so that estimated solution f1(r1) is close to desired solution f(r1).

2. The method of claim 1 wherein said biological tissue is that of a mammal brain and therefore said method is a method for magnetoencephalography.

3. The method of claim 2 wherein said mammal is a human being.

4. The method of claim 1 wherein said biological tissue is that of a mammal heart and therefore said method is a method for magnetocardiography.

5. The method of claim 4 wherein said mammal is a human being.

6. The method of claim 1 wherein magnetic field intensity characteristics includes spatial derivatives of magnetic field intensity.

7. The method of claim 1 wherein said vector-matrix equation g(r2)=H(r1,r2) f(r1)+n(r2) involves convolution of H(r1,r2) and f(r1) and Step (d) is carried-out in the Fourier domain by computing the Fourier transforms of g(r2), and H(r1,r2), and f1(r1) is computed using inverse Fourier transform and inverse filtering in the Fourier domain.

8. An apparatus for measuring magnetic field intensity characteristics around a biological tissue enclosed in a 3D volume space V1, said apparatus comprising a means for measuring magnetic field intensity characteristics of magnetic field generated by an electric current pattern having a 3D spatial density distribution f(r1) in said biological tissue, said means for measuring magnetic field intensity characteristics specifically capable of measuring at a set of points r2 in a 3D volume space V2 that in particular extends substantially along a radial direction pointing away from the approximate center of said biological tissue, and said means for measuring magnetic field intensity characteristics further capable of recording measured data as a function of position r2 of points in said 3D volume space V2 as g(r2).

9. The apparatus of claim 8 which further includes
(a) a means for setting up a vector-matrix equation of type g(r2)=H(r1,r2) f(r1)+n(r2) where H(r1,r2) represents a system matrix that specifies magnetic field intensity at point r2 produced by an electric current density of unit strength located at point r1, and n(r2) represents noise in measured data at point r2; and
(b) a means for solving said vector-matrix equation g(r2)=H(r1,r2) f(r1)+n(r2) and estimating a solution f1(r1) for said 3D image f(r1) using a method that reduces the effect of noise n(r2) so that estimated solution f1(r1) is close to desired solution f(r1).

10. The apparatus of claim 9 wherein said means for solving said vector-matrix equation g(r2)=H(r1,r2) f(r1)+n(r2) includes a means for computing Discrete Fourier transforms and inverse filtering.

11. The apparatus of claim 8 wherein said biological tissue is a brain being imaged in Magentoencephalography.

12. The apparatus of claim 8 wherein said biological tissue is a heart being imaged in Magentocardiography.

13. The apparatus of claim 9 wherein said biological tissue is a brain being imaged in Magentoencephalography.

14. The apparatus of claim 9 wherein said biological tissue is a heart being imaged in Magentocardiography.

15. The apparatus of claim 10 wherein said biological tissue is a brain being imaged in Magentoencephalography.

16. The apparatus of claim 10 wherein said biological tissue is a heart being imaged in Magentocardiography.

17. The apparatus of claim 8 which further includes a computer and a display monitor.

18. The apparatus of claim 8 wherein said means for measuring magnetic field intensity characteristics includes a 3D array of SQUIDS.

19. The apparatus of claim 8 wherein said means for measuring magnetic field intensity characteristics includes a 2D array of SQUIDS which can move to different positions to cover a 3D volume space V2 and measure magnetic field intensity characteristics.
